(19) United States
(12) Patent Application Publication
(10) Pub. No.: US 2016/0114185 A1
(43) Pub. Date: Apr. 28, 2016

(54) Internal UV Treatment Administered Via Endoscopy

(71) Applicant: Lacy Gallaway Mankin, Abilene, TX (US)

(72) Inventor: Lacy Gallaway Mankin, Abilene, TX (US)

(21) Appl. No.: 14/923,711

(22) Filed: Oct. 27, 2015

Related U.S. Application Data
(60) Provisional application No. 62/126,870, filed on Mar. 2, 2015.
(60) Provisional application No. 62/079,098, filed on Nov. 13, 2014.
(60) Provisional application No. 62/074,018, filed on Nov. 1, 2014.
(60) Provisional application No. 62/069,413, filed on Oct. 28, 2014.

(51) Int. Cl. A61N 5/06; A61B 1/04; A61B 1/31; A61B 1/307; A61B 1/273

(52) U.S. Cl. CPC A61N 5/0624; A61N 5/0603; A61B 1/307; A61B 1/2736; A61B 1/31; A61B 1/041; A61N 2005/061; A61N 2005/0608; A61N 2005/0609; A61N 2005/063; A61N 2005/0661

(57) ABSTRACT

A device and method is proposed to enable the treatment of interior of bodily hollow organs and other interior cavities of a body for a number of harmful virus, fungal, and bacterial entities and autoimmune conditions by internal short wavelength ultraviolet light by use of specially equipped endoscope-like devices.

### CROSS REFERENCE TO RELATED APPLICATIONS

This application claims the benefit of U.S. application Ser. No. 62/069,413 filed Oct. 28, 2014 and U.S. application Ser. No. 62/074,018 filed Nov. 1, 2014, and U.S. application Ser. No. 62/079,098 filed Nov. 13, 2014, and U.S. application Ser. No. 62/126,870 filed Feb. 2, 2015.

### BACKGROUND

Ultraviolet germicidal Irradiation (UVGI) Is a disinfection method that uses ultraviolet (UV) light at sufficiently short wavelengths to kill microorganisms, including every existing viral strain that has been scientifically identified. The application of UVGI for disinfection has been an accepted practice since the mid-20th century. It has been used primarily in medical sanitation and sterile work facilities.

Though the method has been used for centuries in a variety of applications, such as food/air, drinking water, and wastewater purification, no method exists to internally treat viral and bacterial infections at this time. Internal treatment in this context can refer to treatment of the upper and lower gastrointestinal tract, the treatment of some hollow organs, and the treatment of some of the structures leading to hollow organs of a body, such as the trachea, esophagus, throat, and urethra. Previous concern for increased potential for the induction of certain cancers appears to be the #1 reason no method exists. The potential for possibly predisposing certain patients to unwanted pathologies, Including cancer, years after UV exposure, is a comparatively trivial drawback in light of effects and usual outcome of the deadly Ebola virus, Staphylococcus infections, C. difficile infections, etc.

The mechanism of action of UVGI method involves utilization of short-wavelengths of ultraviolet radiation (UV-C rays) that are harmful to all microorganisms. It's high efficacy lies in it's ability to destroy the nucleic acids in these organisms, thus disrupting their DNA, leaving them unable to perform vital cellular functions necessary to cause symptoms and disease in or animal tissue.

The mechanism of UVC inactivation of microorganisms is to damage the genetic material in the nucleus of the cell or nucleic acids in the virus. The UVC spectrum, especially the range of 250-270 nm, is strongly absorbed by the nucleic acids of a micro-organism and, therefore, is the most lethal range of wavelengths for microorganisms. This range, with 262 nm being the peak germicidal wavelength, is known as the germicidal spectrum. The light-induced damage to the DNA and RNA of a microorganism often results from the dimerization of pyrimidine molecules. In particular, thymine (which is only found in DNA) produces cyclobutane dimers. When thymine molecules are dimerized, it becomes very difficult for the nucleic acids to replicate and if replication does occur it often produces a defect that prevents the microorganism from being viable.

The particular UV wavelength responsible for this effect Is rare on earth because the atmosphere blocks it. Using a UVGI device in certain environments like circulating air or water systems creates a deadly effect on micro-organisms including: viruses, molds, and bacteria that are present. UVGI is often coupled with a filtration system in these circumstances. In dentistry UV curing lights are used to set the resin filling materials and many types of cements used today. The wavelengths of these dental curing lights ranges from 300-500 nm. An example of this method to treat human ailments was demonstrated over a century ago, only topically however. The 1903 Nobel Prize for Medicine was awarded to Niels Finsen for his use of UV against lupus vulgaris and tuberculosis of the skin.

Using UV light for drinking water disinfection dates back to the year 1910 in France. The prototype plant was taken out of service after only a short time, due to reliability problems. In 1955, UV water treatment systems were applied in Austria and Switzerland; by 1985 about 1,500 plants were in use in Europe. In 1998 it was discovered that protozoa such as cryptosporidum and giardia (also very prevalent in Africa) were more vulnerable to UV light than previously thought; this opened the way to wide-scale use of UV water treatment in North America. By 2001 over 6000 UV water treatment plants were operating in Europe. The cost of UV light has declined significantly over the last few years, making the use of UVGI both highly effective and cost efficient.

In 1878, A. Downes (1851-1938) and T. P Blunt (1842-1929) published a paper describing the sterilization of bacteria exposed to short wavelength light. By 1903, it was discovered that wavelengths around 250 nm were most effective for inactivation of bacteria.

### Method of Operation of Usual Historical UVGI

UV light is electromagnetic radiation with wavelengths shorter than visible light. UV can be separated into various ranges, with short range UV (UVC) considered “germicidal UV”. At certain wavelengths UV is mutagenic to bacteria, viruses and other microorganisms. At a wavelength of 2,537 Angstroms (254 nm)[6] UV will break the molecular bonds within micro-organismal DNA, producing thymine dimers in their DNA thereby destroying them, rendering them harmless or prohibiting growth and reproduction. It Is a process similar to the UV effect of longer wavelengths (UVB) on humans, such as sunburn. Microorganisms have less protection from UV and cannot survive prolonged exposure to it. Usually short duration of between 60-90 seconds of exposure kill almost 100% of the viral or microbial content.

### Effectiveness

The effectiveness of germicidal UV in such an environment depends on a number of certain factors: the length of time a microorganism is exposed to UV, power fluctuations of the UV source that impact the EM wavelength, the presence of obstructions that can protect the micro-organisms from UV, and a micro-organism's ability to withstand UV during its exposure.

In many systems redundancy in exposing microorganisms to UV Is achieved by exposing the antigens repeatedly. This ensures multiple dosages so that the UV is effective against the highest number of microorganisms and will irradiate resistant microorganisms more than once to break them down.

The effectiveness of this form of sterilization is also highly dependent on “line-of-sight” exposure of the microorganisms to the UV light. Environments where design creates obstacles that block the UV light are not as effective (such as the gastrointestinal system, and internal hollow organs). Thus the reason topical application of UV light to the skin cannot expose viral and bacterial contaminants internally. In such an environment the effectiveness Is then reliant on the placement of the UVGI system so that line of sight Is optimum for sterilization. In addition there is the possibility in some applications to use carefully controlled optical clearing techniques (example: pulse-jet lavage techniques) for in situations in which direct line of sight is obstructed by blood or feces.

Thus there is a need and opportunity to address these more severe bacterial and viral infections that occur within a body.

### BRIEF SUMMARY

The approach we propose to address this issue is the use of a flexible and small diameter tube that would insert one or more optical fibers into upper or lower gastrointestinal tract, some hollow organs, such as the lungs, or bladder, or some of the structures leading to hollow organs of a body, such trachea, esophagus, throat, and urethra.

### BRIEF DESCRIPTION OF DRAWINGS

FIG. 1 is an illustration of a cystoscope.

FIG. 2 is an illustration of an endoscope.

FIG. 3 is an illustration of a colonoscope.

### DETAILED DESCRIPTION

In the following detailed description, reference is made to accompanying drawings that illustrate embodiments of the present disclosure. These embodiments are described in sufficient detail to enable a person of ordinary skill in the art to practice the disclosure without undue experimentation. It should be understood, however, that the embodiments and examples described herein are given by way of illustration only, and not by way of limitation. Various substitutions, modifications, additions, and rearrangements may be made without departing from the spirit of the present disclosure. Therefore, the description that follows is not to be taken in a limited sense, and the scope of the present disclosure will be defined only by the final claims.

Endoscopy means “looking inside” and typically refers to looking inside a body for medical reasons using an endoscope, an instrument used to examine the interior of a hollow organ or cavity of a body. Unlike most other medical imaging devices, endoscopes are inserted directly into the organ. There are many different types of endoscopes, and depending on the site in a body and the type of procedure, endoscopy may be performed by a doctor or a surgeon, and the patient may be fully conscious or under a general anesthetic. Endoscope can also refer to using a borescope in technical situations where direct line of-sight observation is not feasible.

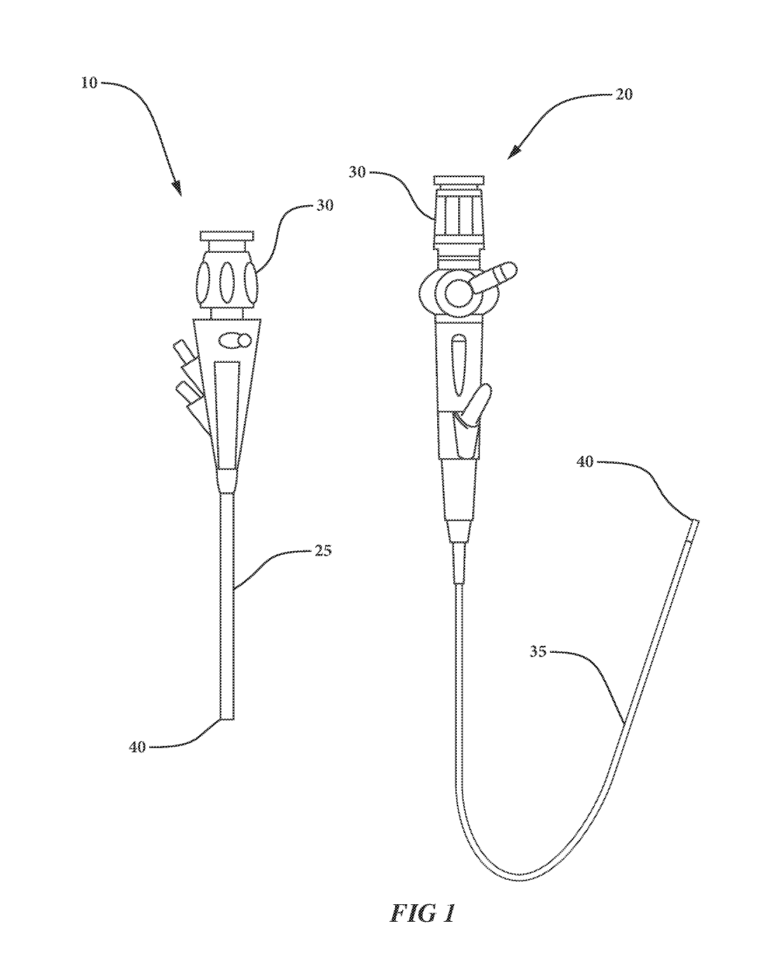

FIGS. 1, 2, and 3 are illustrations of fairly typical endoscope-type devices used in medical practice. These are shown to give examples of the types of devices described herein in this disclosure. There are a wide variety of such endoscope type devices available from a number of suppliers and from each supplier there are a wide variety of designs that are tailored for specific applications. FIGS. 1, 2, and 3 do not cover all of the possibilities.

FIG. 1 is an illustration of an endoscope-like device usually called a cystoscope. The cystoscope is frequently used for endoscopy of the urinary bladder via the urethra. Two types of cystoscopes are presented. Cystoscope 10 is a rigid cystoscope, having a rigid shaft 25 and cystoscope 20 illustrates a flexible cystoscope, having a flexible shaft 35 which may also have a flexible and moveable tip on the distal end 40. The proximal ends 30 of the cystoscope can each have light sources integrated into them to supply the required germicidal UV wavelength frequencies. One special type of thin flexible cystoscope is the ureteroscope, used to traverse beyond the bladder into the ureters, the tubes that carry urine from the kidneys to the bladder. Regardless of the type of cystoscope, light guides within the cystoscopes can be used to deliver the light within the cystoscopes to the distal ends 40 where it can be used to provide controlled dosages of the required UV wavelengths to for example the bladder and the ureters as the cystoscopes are guided through the dosage step.

Some cystoscopes have lenses like a telescope or microscope. These lenses allow a to physician focus on the inner surfaces of the urinary tract. Some cystoscopes use optical fibers that carry an image from the tip of the instrument to a viewing piece at the other end. Cystoscopes range from pediatric to adult and from the thickness of a pencil up to approximately 9 mm and have a light at the tip for illumination. Many cystoscopes have extra tubes to guide other instruments for surgical procedures to treat urinary problems. For purposes of the application described in this disclosure such a cystoscope might have some of these same features but particularly would carry enough additional optical fibers to deliver the required wavelengths with the required intensity to deliver a prescribed dosage of ultraviolet light to the bladder to affect micro-organisms including: viruses, molds, and bacteria that are present.

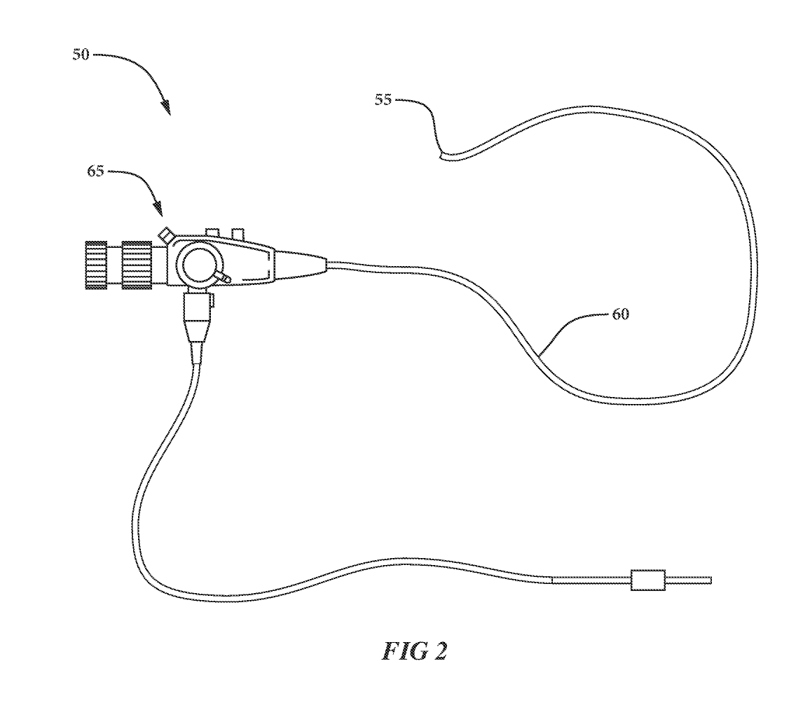

FIG. 2 is an illustration of one type of endoscope. The type shown here is a flexible endoscope, which has a flexible shaft as well as a flexible tip. Endoscopes may include at least a rigid or flexible shaft, a light delivery system, (primarily for illumination), a lens system to transmit an image to a viewer, an eyepiece or camera to receive that image, and additional channels to allow entry of various medical instruments. The proximal end 65 of the endoscope can have a light source integrated to supply the required germicidal UV wavelength frequencies. Light guides within the endoscope can be used to deliver the light within the endoscope to the distal end 55 where it can be used to provide controlled dosages of the required UV wavelengths to the interior of bodily hollow organs and other interior cavities of a body as the endoscope is guided through the dosage step.

For purposes of the application described in this disclosure such an endoscope might have some of these same features but particularly would carry enough additional optical fibers to deliver the required wavelengths with the required intensity to deliver a prescribed dosage of ultraviolet light to the any hollow organ or body cavity to affect micro-organisms including: viruses, molds, and bacteria that are present.

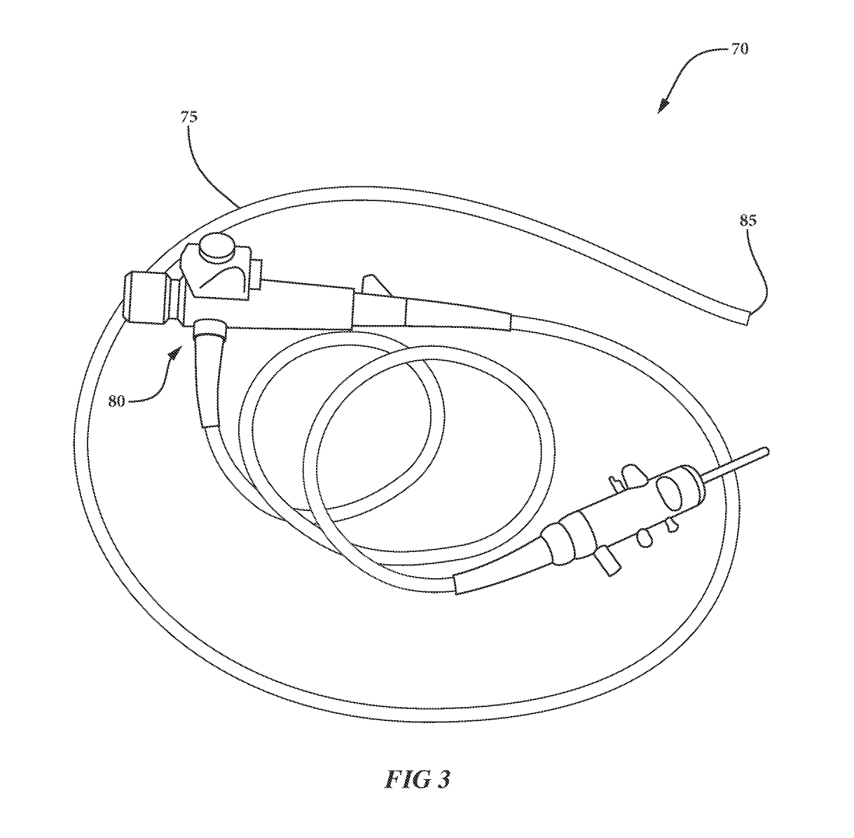

FIG. 3 illustrates one type 70 of colonoscope. These are used for the endoscopic examination of the large bowel and the distal part of the small bowel with a CCD camera or a fiber optic camera on a flexible tube passed through the anus. It can provide a visual diagnosis (e.g. ulceration, polyps) and grants the opportunity for biopsy or removal of suspected colorectal cancer lesions. When used for these purposes colonoscopes might be equipped with sophisticated surgical devices for such removal. Colonoscopes almost always have a flexible shaft 75 for traversing the tortuous path of the colon. The proximal end 80 of the colonoscope can have a light source integrated to supply the required germicidal UV wavelength frequencies. Light guides within the colonoscope can be used to deliver UV light within the colonoscope to the distal end 85 where it can be used to provide controlled dosages of the required UV wavelengths to the interior of colon as the colonoscope is guided through the dosage step.

For purposes of the application described in this disclosure such a colonoscope might have some of these same features but particularly would carry enough additional optical fibers to deliver the required wavelengths with the required intensity to deliver a prescribed dosage of ultraviolet light to the colon and the distal part of the small intestine to affect micro-organisms including: viruses, molds, and bacteria that are present.

It should be noted that this disclosure uses the term endoscope-type device to describe a variety of modalities for probing the interior of bodily hollow organs and other cavities of a body. These include at least esophagogastroduodenoscopy (oesophagus, stomach and duodenum), colonoscopy and sigmoidoscopy (large intestine/colon), endoscopic retrograde cholangiopancreatography (bile duct), rectoscopy or proctoscopy (rectum), rhinoscopy (nose), bronchoscopy (lower respiratory tract), otoscopy (ear), cystoscopy (uninary tract), hysteroscopy (uterus), falloposcopy (fallopian tubes).

Not all endoscope-type devices have long structures that are inserted into bodies. Capsule endoscopy is used to examine parts of the gastrointestinal tract that cannot be seen with other types of endoscopy. The capsule is the size and shape of a pill and usually contains a tiny camera. After a patient swallows the capsule, it takes pictures of the inside of the gastrointestinal tract. The primary use of capsule endoscopy is to examine areas of the small intestine that cannot be seen by other types of endoscopy, but unlike other endoscope-type devices colonoscopy it cannot ordinarily treat pathology that may be discovered. Capsule endoscopy transfers the captured images wirelessly to an external receiver worn by the patient using one of appropriate frequency bands. The collected images are then transferred to a computer for diagnosis, review and display. A transmitted radio-frequency signal can be used to accurately estimate the location of the capsule and to track it in real time inside a body and gastrointestinal tract.

If provided with an internal source of UV germicidal frequency light that could be turned on and off when needed a capsule endoscope could be used to provide controlled dosages of required UV wavelengths to the interior of normally inaccessible regions of the gastrointestinal tract such as the smaller intestine as the capsule colonoscope is guided through the dosage step.

In addition some normally closed body cavities can be accessed and treated with ultraviolet light of the appropriate frequency through small incisions. Laparoscopy (abdominal or pelvic cavity), arthroscopy (interior of joints), and thoracoscopy and mediastinoscopy (organs of the chest).

The endoscope-type devices described herein could deliver needed short wavelength germicidal UV light through the entire gastrointestinal tract and address the “line-of-sight” problem. In this disclosure we use the broadest definition of the gastrointestinal tract that includes all structures between the mouth and throat region, and the anus, and the potential treatment of any part of that tract. An example device could be an endoscope-like device that already has the high flexibility and small diameter for insertion into the gastrointestinal tract. For explanatory purposes this type of device will be referred to as an endoscope or endoscope type device, although it could have other names, including at least the terms colonoscope, or a cystoscope.

The approach also has the potential to treat other hollow organs, such as the lungs or bladder.

In one embodiment this endoscope-like device could be inserted rectally, giving the correct wavelength UV rays directed access to the virally infected tissue lining the colon.

In another embodiment this endoscope-like device could be inserted through the nasal cavity, giving the correct wavelength UV rays directed access to the virally infected tissue lining the upper gastrointestinal tract.

In another embodiment this endoscope-like device could be inserted through the mouth, giving the correct wavelength UV rays directed access to the virally infected tissue lining the upper gastrointestinal tract.

In another embodiment this endoscope-like device could access hollow organs such as the bladder or the lungs, as well as some of the structures leading to those organs such as the trachea, esophagus, throat, or urethra.

In any of these embodiments a light delivery system can be used to deliver the required UV light frequencies. The light source may be outside of a body and the light may be directed via an optical fiber system.

In another embodiment an endoscopic tube can be inserted and guided through the gastrointestinal tract and the outside tubing removed leaving the thin optical fiber ready for UV exposure. An example therapy could be 60 seconds of UV exposure time repeated three times daily. After the exposure time the filament can be removed from the body and the optical fiber material removed from the source and properly disposed of.

In another embodiment a small UV germicidal bulb could be attached to the endoscopic like device to deliver the UV wavelength as the endoscope-like device travels through the treated region.

Such an endoscopic-like device can further include a lens system that transmits the image from the objective lens to the viewer, typically a relay lens system or a bundle of optical fibers in the case of a fiberscope. Visualization may be to an eyepiece or a video scope with no eyepiece, in which a camera transmits an image to a screen for image capture.

It is recognized that there are a number of combinations of endoscopic like devices, optical fibers, light sources, and insertion/removal methodologies that can be potentially applied to deliver the needed wavelengths to the appropriate locations in the gastrointestinal tract as well into other hollow organs and structures that may require treatment.

There are a wide variety of optical fibers created for multiple applications. An optical fiber is a flexible, thin fiber made of extruded glass or plastic. It can function as a waveguide, or “light pipe”, to transmit light between the two ends of the fiber. Optical fibers are widely used in fiber-optic communications; and can also be used for illumination, and can be used individually or in bundles. Specially designed fibers are used for a variety of other applications, including sensors and fiber lasers.

Not all fibers are designed as light pipes. There are special edge-emitting fibers that are designed to “leak” light along the fiber. And such fibers are candidates for this methodology. Such side or edge emitters could be designed to leak at a uniform rate or could be designed with intermittent locations along the fiber in which the fiber outer cladding is significantly reduced to allow UV light to escape.

Ultraviolet (UV) irradiation is electromagnetic irradiation with a wavelength (100-400 nm) shorter than that of visible light (400-700 nm), but longer than x-rays (<100 nm). UV irradiation is divided into four distinct spectral areas including vacuum UV (100-200 nm), UVC (200-280 nm), UVB (280-315 nm) and UVA (315-400 nm), The mechanism of UVC inactivation of microorganisms is to damage the genetic material in the nucleus of the cell or nucleic acids in the virus. The UVC spectrum, especially the range of 250-270 nm, is strongly absorbed by the nucleic acids of a micro-organism and, therefore, is the most lethal range of wavelengths for microorganisms. This range, with 262 nm being the peak germicidal wavelength, is known as the germicidal spectrum.

This disclosure anticipates the use of any of these possible combinations. The ultraviolet wavelengths normally considered as germicidal are in the 250-270 nm range although it is anticipated that nearby wavelengths could have efficacy.

The optical fiber could be carried along inside the endoscope-like device or attached to the endoscope-like device.

The method of application can vary depending on the type of fiber used. With a fiber designed as a light pipe the UV wavelength might be delivered predominately at the end of the fibers and the method then might be to progress the endoscope-like device in a programmed manner through the parts of a body to be treated at a rate designed to deliver the needed dosage.

### Example Dosages

An ex vivo study was carried out by Taylor et at, to investigate the use of UVC irradiation (254 nm) for the prophylaxis of surgical site infections. The authors modeled a ‘clean’ surgical wound lightly contaminated with airborne bacteria by using agar, ovine muscle and ovine adipose tissue, respectively. It was found that airborne bacteria were inhibited more rapidly and more completely on agar than on muscle. A coating of blood over the micro-organisms on muscle substantially reduced the effectiveness of UVC. At an irradiance of 1.2 mW/cm² calculated at the lamp aperture, 1 min UVC irradiation time reduced bacterial colony forming units (CFUs) by 99.1% on agar, 97.1% on muscle (p=0.046) and 53.5% on muscle coated with blood (p<0.001). The combination of pulsed jet lavage and UVC was tested with the intention to remove the blood coated over the bacteria prior to UVC irradiation. The bacterial CFUs were reduced by 97.7% with the combination of pulsed jet lavage and UVC.

In the case of Ebola the rays could quickly destroy the rapidly replicating Ebola virus thus diminishing symptoms and, possibly even preventing the diarrhea and vomiting from beginning. With the rapid loss of bodily fluids being the main factor resulting In death, the elimination or decrease in GI symptoms could buy time for the patient to allow the patient's body to fight the infection.

Very few drawbacks for this method have been documented, and the solution to these drawbacks is extremely simple. The following have been noted in studies:

Sterilization is often misquoted as being achievable. While it is theoretically possible in a controlled environment, it is very difficult to prove and companies offering this service as to avoid legal use the term “disinfection”. Specialist companies will often advertise a certain log reduction e.g., 99.9999% effective, instead of sterilization. This takes into consideration a phenomenon known as light and dark repair (photo reactivation and base excision repair, respectively) In which the DNA in the bacterium will fix itself after being damaged by UV light.

### Inactivation of Microorganisms

The degree of inactivation by ultraviolet radiation is directly related to the UV dose applied to the water. The dosage, a product of UV light intensity and exposure time, is usually measured In micro joules per square centimeter, or alternatively as microwatt seconds per square centimeter (μW·s/c^{m}2). Dosages for a 90% kill of most bacteria and virus range from 2,000 to 8,000 μW·s/cm2. Dosage for larger parasites such as Cryptosporidiurn require a lower dose for inactivation. As a result, the U.S. Environmental Protection Agency has accepted UV disinfection as a method for drinking water plants to obtain Cryptosporidium, Giardia or virus inactivation credits. For example, for one-decimal-logarithm reduction of Cryptosporidium, a minimum dose of 2,500 μW-s/cm2 i5 required based on the U.S. EPA UV Guidance Manual published In 2006.

The U.S. EPA has published UV dosage guidelines. Our design can be disposable and/or non-disposable. With our product following the dosage guidelines, ease of disbursement, low cost, ease of use (very little user/administrator training necessary. and no potential for harm to the administrators (Goggles or shields should be worn as a precaution), it has high potential as an effective Intervention in internal viral or bacterial infections. We anticipate the possible treatment of Ebola, C. difficile, Staphylococcus, and many others.

### Applications

The potential effective treatment of the large portions of the gastrointestinal tract using the proposed system has a number of applications in terms of patient treatment. The treatment of Ebola has been mentioned, but there are a number of other serious viral and/or bacterial infections that can get out of control in the human gastrointestinal tract and in some hollow organs. The average human gastrointestinal tract is home too as many as 1,000 species of microorganisms. Most of them are harmless—or even helpful—under normal circumstances. But when something upsets the balance of these organisms in the gut, otherwise harmless bacteria can grow out of control and lead to terrible sickness. One of the worst offenders is a bacterium called Clostridium difficile (C. difficile, or C. diff). As the bacteria overgrow they release toxins that attack the lining of the intestines, causing a condition called Clostridium difficile colitis. Though relatively rare compared to other intestinal bacteria, C. difficile is one of the most important causes of infectious diarrhea in the U.S. Hospital stays from C. difficile infections have tripled in the last decade and one estimate is that C. difficile infections cost at least $1 billion in extra health care costs in the U.S. only. There have been major outbreaks in other countries also.

C. difficile is one of the most commonly transmitted infections among healthcare workers and patients in facilities including nursing homes and hospitals. It is highly contagious, very aggressive, and the bacteria are more difficult to extinguish on surfaces when compared to other bacterial and viral components. However, C. diff should be effectively treated with the wavelength of UV light delivered via the optical fiber filament of the devices proposed.

The occurrence of an infection of C. difficile is often a result of exposure to long term or high dose of antibiotics used to treat other medical ailments locally and systemically in the patient. The antibiotics administered kill the targeted unwanted bacteria in the patient's system, but unfortunately they also kill a high percentage of the “good” bacteria that reside as “normal flora” in the gut to maintain and support the overall immune system. The unwanted C. Diff bacterial count is thus allowed to multiply substantially and cause infectious diarrhea. UV endoscopic therapy could also kill both “good and bad bacteria” residing in the gut. However, the probiotic administration of concurrent high and frequent doses of the “good” bacteria will encourage the increase of the good bacteria. Daily probiotics supplements of this “good bacteria” are taken by millions of Americans daily as an immune or digestive supplement. Lactobacillus is one of the most commonly well known “helpful” GI bacteria that promote healthy digestion and supports immune health. These particular bacteria would be given to patients (in addition to a surplus of others) treated endoscopically with UV treatment for C. Diff and or other bacterial and viral infections to promote the recurrent growth of helpful bacteria. In addition fecal transplantation is often used to re-cultivate helpful bacteria.

It should be noted that UV light is currently used as an external treatment by dermatologists therapeutically for the active symptoms of psoriasis and eczema. Though autoimmune conditions are scientifically classified as diseases not resulting from bacterial or viral components, their cause is usually unknown. However, the symptoms are often severe. UV light administered by dermatologists to localized areas of severe ulceration or other symptoms active on the skin that are associated with autoimmune disorders. The ulcerations, irritation, diarrhea and inflammation occurring in the GI tract in most all autoimmune disorders resemble the visible external lesions present on the skin. UV light has proven effective in multiple aspects of the treatment for dermatological autoimmune conditions thus increasing the likelihood for the UV to have similar efficacy in the GI tract when introduced in these conditions.

Every case would vary depending on the patient's medical specifications. Each individual's treatment (wavelength, frequency of administration, length of exposure, location of intestine exposed, etc.) would depend on input from medical specialists in various areas, including oncology, internal medicine, and autoimmune disorders etc. who were familiar with the patient's case. Thus the wavelength, localized administration to ulcerated areas only, or areas of greatest concern within the entire gastrointestinal tract could be tapered or altered to the patient's specific needs of indications of use.

The treatment of a number of serious viral and/or bacterial infections as well as some autoimmune conditions are all potential applications of the devices of this disclosure and all are anticipated.

### Veterinary Applications

The use of endoscope-like devices equipped with the ability to provide controlled dosages of ultraviolet “germicidal” wavelengths of light could easily apply to many applications in veterinary medicine. Any of the types of endoscope-type devices described in this disclosure could be designed to carry enough additional optical fibers to deliver the required wavelengths with the required intensity to deliver a prescribed dosage of ultraviolet light to the any hollow organ or body cavity in many animals to effectively treat micro-organisms including: viruses, molds, and bacteria that are present.

Although certain embodiments and their advantages have been described herein in detail, it should be understood that various changes, substitutions and alterations could be made without departing from the coverage as defined by the appended claims. Moreover, the potential applications of the disclosed techniques is not intended to be limited to the particular embodiments of the processes, machines, manufactures, means, methods and steps described herein. As a person of ordinary skill in the art will readily appreciate from this disclosure, other processes, machines, manufactures, means, methods, or steps, presently existing or later to be developed that perform substantially the same function or achieve substantially the same result as the corresponding embodiments described herein may be utilized. Accordingly, the appended claims are intended to include within their scope such processes, machines, manufactures, means, methods or steps.

1. An endoscope-like device for delivery of ultra violet (UV) light in controlled dosages to the interior of bodily hollow organs and other interior cavities of a body comprising:
a. a proximal end;
b. a distal end for insertion into the interior of bodily hollow organs and other interior cavities of the body;
c. a light source integrated into the proximal end of the endoscope-like device;
d. one or more light guides to collect the light from the light source and deliver the light within the endoscope-like device to the distal end of the endoscope-like device;
e. wherein the light source emits and the one or more light guides delivers to the distal end of the endoscope-like device UV irradiation with a wavelength of 100 to 400 nanometers.

2. The endoscope-like device for delivery of ultra violet (UV) light in controlled dosages to the interior of bodily hollow organs and other interior cavities of a body of claim 1 wherein the endoscope-like device is a colonoscope for delivery of the UV light to regions of the colon.

3. The endoscope-like device for delivery of ultra violet (UV) light in controlled dosages to the interior of bodily hollow organs and other interior cavities of a body of claim 1 wherein the endoscope-like device is a cystoscope for delivery of UV light to the urethra.

4. The endoscope-like device for delivery of ultra violet (UV) light in controlled dosages to the interior of bodily hollow organs and other interior cavities of a body of claim 1 wherein the endoscope-like device is an endoscope for delivery of UV light to the upper gastrointestinal tract.

5. The endoscope-like device for delivery of ultra violet (UV) light in controlled dosages to the interior of bodily hollow organs and other interior cavities of a body of claim 1 wherein the light source emits and the light guide delivers to the distal end of the endoscope-like device UV irradiation with a wavelength of 250 to 270 nanometers.

6. The endoscope-like device for delivery of ultra violet (UV) light in controlled dosages to the interior of bodily hollow organs and other interior cavities of the body of claim 1 wherein the interior of bodily hollow organs and other interior cavities of the body refers to the human body.

7. The endoscope-like device for delivery of ultra violet (UV) light in controlled dosages to the interior of bodily hollow organs and other interior cavities of a body of claim 1 wherein the interior of bodily hollow organs and other interior cavities of the body refers to animal bodies.

8. A method for delivery of ultra violet (UV) light in controlled dosages to the interior of bodily hollow organs and other interior cavities of a body comprising:
a. providing an endoscope-like device with a proximal end and a distal end;
b. providing a light source integrated into the proximal end of the endoscope type device;
c. providing one or more light guides to collect the light from the light source and deliver the light within the endoscope-like device to the distal end of the endoscope-like device;
d. guiding the endoscope-like device in a programmed manner through designated interior parts of bodily hollow organs and other interior cavities the body to deliver needed dosages of light from the light source;
e. wherein the light source emits and the one or more light guides delivers to the distal end of the endoscope-like device UV irradiation with a wavelength of 100 to 400 nanometers.

9. The method for delivery of ultra violet (UV) light in controlled dosages to the interior of bodily hollow organs and other interior cavities of a body of claim 8 wherein the endoscope-like device is a colonoscope for delivery of the UV light to regions of the colon.

10. The method for delivery of ultra violet (UV) light in controlled dosages to the interior of bodily hollow organs and other interior cavities of a body of claim 8 wherein the endoscope-like device is a cystoscope for delivery of UV light to a urethra.

11. The method for delivery of ultra violet (UV) light in controlled dosages to the interior of bodily hollow organs and other interior cavities of a body of claim 8 wherein the endoscope-like device is an endoscope for delivery of UV light to an upper gastrointestinal tract.

12. The method for delivery of ultra violet (UV) light in controlled dosages to the interior of bodily hollow organs and other interior cavities of a body of claim 8 wherein the light source emits and the light guide delivers to the distal end of the endoscope-like device UV irradiation with a wavelength of 250 to 270 nanometers.

13. The method for delivery of ultra violet (UV) light in controlled dosages to the interior of bodily hollow organs and other interior cavities of a body of claim 8 wherein the interior of bodily hollow organs and other interior cavities of the body refers to the human body.

14. The method for delivery of ultra violet (UV) light in controlled dosages to the interior of bodily hollow organs and other interior cavities of a body of claim 8 wherein the interior of bodily hollow organs and other interior cavities of the body refers to animal bodies.

15. A capsule endoscope-like device for delivery of ultra violet (UV) light in controlled dosages to regions of the small intestine comprising:
a. a light source integrated into the capsule endoscope-like device;
b. wherein the light source emits UV irradiation with a wavelength of 100 to 400 nanometers.

16. The capsule endoscope-like device for delivery of ultra violet (UV) light in controlled dosages to regions of the small intestine of claim 15 wherein the light source emits device UV irradiation with a wavelength of 250 to 270 nanometers.

17. A method for delivery of ultra violet (UV) light in controlled dosages to regions of the small intestine comprising:
a. providing a capsule endoscope-like device;
b. providing a light source integrated into the capsule endoscope-like device;
c. wherein the light source emits device UV irradiation with a wavelength of 100 to 400 nanometers.

18. The method for delivery of ultra violet (UV) light in controlled dosages to regions of the small intestine of claim 17 wherein the light source emits device UV irradiation with a wavelength of 250 to 270 nanometers.
